(19) United States
(12) Patent Application Publication
(10) Pub. No.: US 2014/0363780 A1
(43) Pub. Date: Dec. 11, 2014

(54) ORAL CARE COMPOSITIONS

(71) Applicants: Joe Vazquez, Hamilton, NJ (US); Dandan Chen, Edison, NJ (US); Harsh M. Trivedi, Hillsborough, NJ (US); Ammanuel Mehreteab, Piscataway, NJ (US); Laurence Du-Thumm, Princeton, NJ (US); Gregory Szewczyk, Flemington, NJ (US); Zhigang Hao, Bridgewater, NJ (US)

(72) Inventors: Joe Vazquez, Hamilton, NJ (US); Dandan Chen, Edison, NJ (US); Harsh M. Trivedi, Hillsborough, NJ (US); Ammanuel Mehreteab, Piscataway, NJ (US); Laurence Du-Thumm, Princeton, NJ (US); Gregory Szewczyk, Flemington, NJ (US); Zhigang Hao, Bridgewater, NJ (US)

(73) Assignee: BRIGHAM YOUNG UNIVERSITY, Provo, UT (US)

(21) Appl. No.: 14/339,342

(22) Filed: Jul. 23, 2014

Related U.S. Application Data
(63) Continuation-in-part of application No. 14/364,283, filed on Jul. 29, 2014.

(51) Int. Cl. A61K 8/63; A61C 19/06; A61C 7/14; A61C 7/20; A61Q 11/00; A61C 7/08

(52) U.S. Cl. CPC A61K 8/63; A61Q 11/00; A61C 7/08; A61C 7/14; A61C 7/20; A61C 19/063; A61K 2800/70
USPC 433/6; 424/49; 424/52; 424/401; 552/554; 433/2; 433/8; 433/20

(57) ABSTRACT

Described herein are aqueous and substantially non-aqueous oral compositions comprising a cationic steroidal compound and methods of making and using the same.

### CROSS-REFERENCE TO RELATED APPLICATIONS

This application is a continuation-in-part of U.S. patent application Ser. No. 14/364,283, filed Jun. 10, 2014, which is a 371 national phase of International Application No. PCT/US2011/066482, filed Dec. 21, 2011, the disclosures of which are incorporated herein by reference in their entirety.

### BACKGROUND

Ceragenins are cationic steroid antimicrobials that are synthetically produced from a sterol backbone.

Quaternary ammonium compounds are known to have antibacterial activity and their use in oral care is also known. However, oral care products containing a combination of a quaternary ammonium compound together with a cationic steroidal compound have heretofore been unknown.

### SUMMARY

Some embodiments of the present invention provide an aqueous oral care composition comprising a cationic steroidal compound. Some embodiments of the present invention provide a substantially non-aqueous oral care composition comprising a cationic steroidal compound.

Other embodiments provide methods of treating a disease or condition of the oral cavity comprising administering a composition according to any of the foregoing claims to the oral cavity of a subject in need thereof.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Following is a brief description of the appended drawings.

FIG. 1 is a chart that shows the results of plaque treatment with CSA solutions compared to a chlorhexidine containing control.

FIG. 2 is a chart that shows the results of plaque treatment with CSA solutions compared to chlorhexidine and cetylpyridinium chloride containing controls.

FIG. 3 is a chart that shows reduction in levels of volatile sulfur compounds (VSCs) generated by salivary bacteria after treatment with CSA-13.

### DETAILED DESCRIPTION

Some embodiments of the present invention provide an aqueous oral care composition comprising a cationic steroidal compound. Some embodiments of the present invention provide a substantially non-aqueous oral care composition comprising a cationic steroidal compound. Some embodiments provide a composition of the compound of Formula (I):

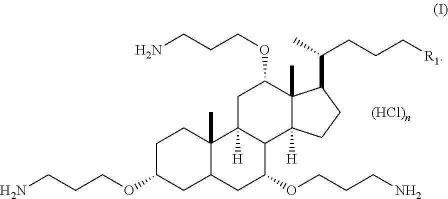

As used herein, the term “aqueous” refers to a free water content of at least about 40%, by weight. As used herein, the phrase “substantially non-aqueous” refers to a free water content of no more than about 10%, by weight.

In some embodiments, the compositions comprise from about 40% to about 97%, by weight, free water. In some embodiments, the compositions comprise greater than about 50%, by weight, free water. In some embodiments, the compositions comprise from about 50% to about 90%, by weight, free water. In some embodiments, the compositions comprise from about 60% to about 85%, by weight, free water. In some embodiments, the compositions comprise from about 73% to about 83%, by weight, free water. Some embodiments comprise about 74%, about 75%, about 76%, about 77%, about 78%, about 79%, about 80%, about 81% or about 82%, by weight, free water.

In some embodiments, the compositions comprise from about 1 to about 10%, by weight, free water. In some embodiments, the compositions comprise from about 1 to about 8%, by weight, free water. In some embodiments, the compositions comprise from about 1 to about 7%, by weight, free water. In some embodiments, the compositions comprise from about 1% to about 6%, by weight, free water. In some embodiments, the compositions comprise from about 1% to about 5%, by weight, free water. In some embodiments, the compositions comprise from about 1% to about 4%, by weight, free water. In some embodiments, the compositions comprise from about 1% to about 3%, by weight, free water. In some embodiments, the compositions comprise from about 1% to about 2%, by weight, free water. Some embodiments comprise about 1%, about 2%, about 3%, about 4%, about 5%, about 6%, about 7%, about 8%, about 9%, or about 10%, by weight, free water.

In some embodiments, the oral care formulation is an aqueous formulation. In some embodiments, the oral care formulation is selected from the group consisting of mouthwash, mouthrinse, toothpaste, mousse, foam, mouth spray. In some embodiments, the composition is a mouthwash. In some embodiments, the composition is substantially free of ethanol.

In some embodiments, the oral care formulation is a substantially non-aqueous formulation. In some embodiments, the oral care composition is a formulation selected from the group consisting of lozenge, tablet, dental implement, orthodontic device, flexible strip, or chewing gum.

In some embodiments, the cationic steroidal compound is carried by a flexible strip, the strip further comprising a first side and a second side, and wherein at least one side comprises an adhesive. In some embodiments, the strip comprises a matrix comprising a fast-dissolving material. In some embodiments, the strip comprises a slow-release mucoadhesive device. In some embodiments, the cationic steroidal compound is in an orthodontic device. In some embodiments, the orthodontic device is selected from the group consisting of a removable retainer, a temporary retainer, a permanent retainer, braces brackets, braces wires, braces fasteners, a bridge, and tooth spacers.

In some embodiments, the cationic steroidal compound is a compound of Formula (I):

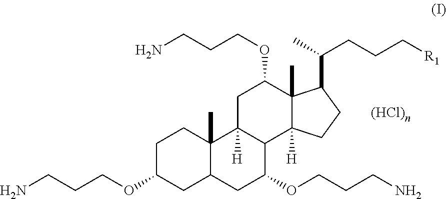

wherein R₁ is selected from —OH and NH—R₂, wherein R₂ is C₂-C₁₄ alkyl, C₂-C₁₄ alkenyl or C₂-C₁₄ akynyl, and n is 3 or 4.

Some embodiments provide a composition wherein the compound of Formula (I), is selected from a compound of Formula (II):

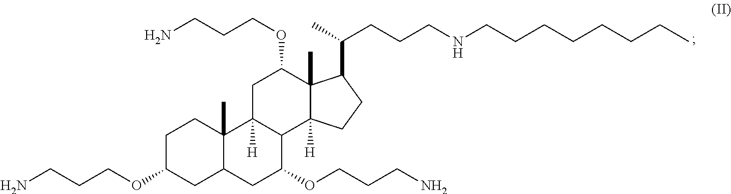

a compound of Formula (III):

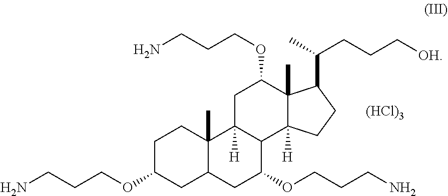

In some embodiments, the cationic steroidal compound is a compound of Formula (II):

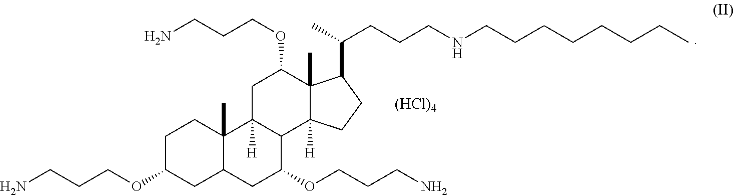

In some embodiments, the cationic steroidal compound has a molecular weight of from about 500 to about 1000. In some embodiments, the cationic steroidal compound has a molecular weight of from about 650 to about 850.

In some embodiments, the cationic steroidal compound is present at a concentration of from about 0.01% to about 0.1%, by weight, of the composition. In some embodiments, the cationic steroidal compound is present at a concentration of about 0.05%, by weight, of the composition.

In some embodiments, a quaternary ammonium compound may be included and can be selected from: benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide. In some embodiments, the quaternary ammonium compound comprises cetylpyridinium chloride.

Quaternary ammonium compounds are a group of ammonium salts in which organic radicals have been substituted for all four hydrogens of the original ammonium cation. They have a central nitrogen atom which is joined to four organic radicals and one acid radical. Examples of quaternary ammonium compounds suitable for use in the instant invention further include other benzalkonium or benzethonium halides, including, but not limited to, benzalkonium or benzethonium bromide or fluoride, cetyl p alkylamidopropalkonium chloride, behenalkonium chloride, behentrimonium methosulphate, behenamidopropylethyldimonium ethosulphate, stearalkonium chloride, olealkonium chloride, cetrimonium chloride, dequalinium chloride, N-myristyl-N-methyl-morpholinium methyl sulfate, poly[N-[3-(dimethylammonio)propyl]-N′-[3-(ethyleneoxyethelenedimethyl-ammoinio)propyl]urea dichloride], alpha-4-[1-tris(2-hydroxyethyl)ammonium chloride-2-butenyl]-omega-tris(2-hydroxyethyl)ammonium chloride, and poly[oxyethylene(dimethyliminio)ethylene(dimethyliminio)-ethylene dichloride].

In some embodiments, the quaternary ammonium compound is present at a concentration of from about 0.01% to about 0.1%, by weight, of the composition. In some embodiments, the quaternary ammonium compound is present at a concentration of about 0.05%, by weight, of the composition.

In some embodiments, the cationic steroidal compound and the quaternary ammonium compound can be present in a 1:1 ratio, based on their respective concentrations, by weight, in the composition.

Some embodiments of the present invention further comprise a fluoride ion source, wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof. In some embodiments, the fluoride ion source comprises sodium fluoride.

Other embodiments provide a method of treating a disease or condition of the oral cavity comprising administering a composition according to any of the foregoing claims to the oral cavity of a subject in need thereof. In some embodiments, the disease or condition of the oral cavity is an inflammatory disease or condition. In some embodiments, the disease or condition is selected from gingivitis, periodontitis, and caries.

In some embodiments, the present invention provides methods of treating an inflammatory condition of the oral cavity, comprising administering a composition comprising a cationic steroidal compound to the oral cavity of a subject in need thereof.

Some embodiments provide a method of treating oral malodor comprising administering an effective amount of a composition of the present invention to the oral cavity of a subject in need thereof. In some embodiments, the compositions of the present invention reduce volatile sulfur compounds (VSC) generated from odor producing salivary bacteria.

Some embodiments provide a method of treating a disease or condition of the oral cavity comprising identifying a subject with a disease or condition of the oral cavity and administering a composition comprising a cationic steroidal compound to the oral cavity of the subject in need thereof. In some embodiments, the disease or condition of the oral cavity is selected from the group consisting of gingivitis, periodontitis, and caries.

Some embodiments provide a method treating an inflammatory condition of the oral cavity comprising identifying a subject with an inflammatory condition of the oral cavity and administering a composition comprising a cationic steroidal compound to the oral cavity of a subject in need thereof.

Some embodiments provide a method of treating halitosis comprising identifying a subject with halitosis and administering a composition comprising a cationic steroidal compound to the oral cavity of a subject in need thereof. In some embodiments, there is a reduction of volatile sulfur compounds in oral cavity of a subject in need thereof.

In some embodiments, the levels of at least one of hydrogen sulfide, methyl mercaptan, and dimethyl sulfide in the oral cavity are reduced from about 20% to about 99%. In some embodiments, the levels are reduced from about 30% to about 99%. In some embodiments, the levels are reduced from about 40% to about 99%. In some embodiments, the levels are reduced from about 50% to about 99%. In some embodiments, the levels are reduced from about 60% to about 99%. In some embodiments, the levels are reduced from about 70% to about 99%. In some embodiments, the levels are reduced from about 80% to about 99%. In some embodiments, the levels are reduced from about 95% to about 99%. In some embodiments, the levels are reduced from about 95% to about 99%.

In some embodiments, the levels of at least one of hydrogen sulfide, methyl mercaptan, or dimethyl sulfide in the oral cavity are reduced by at least about 20%. In some embodiments, the levels are reduced by at least about 25%. In some embodiments, the levels are reduced by at least about 30%. In some embodiments, the levels are reduced by at least about 35%. In some embodiments, the levels are reduced by at least about 40%. In some embodiments, the levels are reduced by at least about 45%. In some embodiments, the levels are reduced by at least about 50%. In some embodiments, the levels are reduced by at least about 55%. In some embodiments, the levels are reduced by at least about 60%. In some embodiments, the levels are reduced by at least about 65%. In some embodiments, the levels are reduced by at least about 70%. In some embodiments, the levels are reduced by at least about 75%. In some embodiments, the levels are reduced by at least about 80%. In some embodiments, the levels are reduced by at least about 85%. In some embodiments, the levels are reduced by at least about 90%. In some embodiments, the levels are reduced by at least 95%. In some embodiments, the levels are reduced by at least about 99%.

In some embodiments, the reduction in volatile sulfur compounds in the oral cavity of a subject in need thereof is not accomplished by oxidation.

Some embodiments provide a method of preventing halitosis comprising identifying a subject at risk for developing halitosis and administering a composition comprising a cationic steroidal compound to the oral cavity of a subject in need thereof.

Some embodiments provide a composition of a compound of Formula (I):

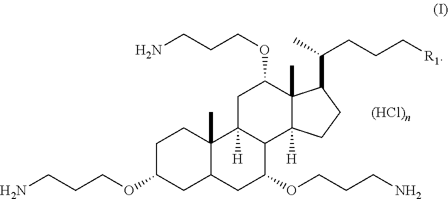

In some embodiments, R₁ is selected from —OH and NH—R₂. In some embodiments, R₂ is selected from C₂-C₁₄ alkyl, C₂-C₁₄ alkenyl or C₂-C₁₄ akynyl. In some embodiments, n is 3 or 4. In some embodiments, R₁ is —OH. In some embodiments, R₁ is NH—R₂. In some embodiments, R₂ is C₂-C₁₄ alkyl. In some embodiments, R₂ is C₂-C₁₄ alkenyl. In some embodiments, R₂ is C₂-C₁₄ akynyl. In some embodiments, n is 3. In some embodiments, n is 4.

The present inventors have discovered that a combination of a cationic steroidal compound (e.g., a ceragenin) and a quaternary ammonium compound (e.g. cetylpyridinium chloride) provides an unexpectedly enhanced antimicrobial activity.

As used herein the term “a ceraginin” includes combinations of ceraginins and “a quaternary ammonium compound” includes combinations of quaternary ammonium compounds.

Ceragenins are cationic steroid antibiotics (CSAs). They can be synthetically produced and are small molecule chemical compounds consisting of a sterol backbone with amino acids and other chemical groups attached to them. These compounds have a net positive charge that is electrostatically attracted to the negatively charged cell membranes of certain viruses, fungi and bacteria. CSAs have a high binding affinity for such membranes and are able to rapidly disrupt the target membranes leading to rapid cell death.

The cationic properties of ceraginins mimic the cationic charge of peptides. Ceraginins contemplated to be useful in the present invention are disclosed in U.S. Pat. No. 6,767,904. In one embodiment the ceraginin is a compound of Formula (II):

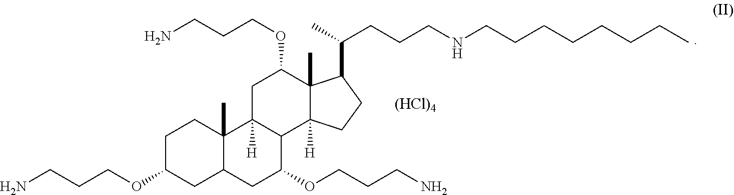

The biological activity of the ceragenin and quaternary ammonium compounds can be determined by standard methods known to those of skill in the art, such as the “minimum inhibitory concentration (MIC)” assay, whereby the lowest concentration at which no change in optical density (OD) is observed for a given period of time is recorded as MIC. When the compound alone is tested against a control that lacks the compound, the antimicrobial effect of the compound alone is determined.

Alternatively, “fractional inhibitory concentration (FIC)” is also useful for determination of synergy between the compounds. The use of the terms, “synergistic” and “synergy,” are used in the present invention to mean an antibacterial effect created from the application of two or more compounds to produce an antibacterial effect that is greater than the sum of the antibacterial effects produced by the application of the individual compounds. The FIC procedure permits determination of synergistic effects of a combination of the compounds. FICs can be performed by checkerboard titrations of one compound in one dimension of a microtiter plate, and of the other compound in the other dimension, for example. The FIC is calculated by looking at the impact of one compound on the MIC of the other and vice versa. An FIC of one indicates that the influence of the compounds is additive and an FIC of less than one indicates synergy. In some embodiments, an FIC of less than 0.7 indicates synergy between the compounds being evaluated.

As used herein, FIC can be determined as follows:

FIC=A+B
where A=(MIC of combination X+Y/(MIC of X alone)
   B=(MIC of combination X+Y/(MIC of Y alone)

The combination of antimicrobial compounds of the present invention is effective against a wide variety of microorganisms such as oral bacteria. Examples of such bacteria include, but are not necessarily limited to, Actinomyces viscosus, Streptococcus mutans, Porphyromonas gingivalis, Fusobacterium nucleatum, and the like.

In some embodiments, the compositions of the present invention are able to provide the antimicrobial effect after about 30 seconds. This ability is particularly advantageous for the embodiments of the present invention which are in the form of a mouthwash, as 30 seconds corresponds to the ordinary duration of use for a mouthwash.

In some embodiments, the compositions comprise a buffering agent, e.g., sodium phosphate buffer (e.g., sodium phosphate monobasic and disodium phosphate).

In some embodiments, the compositions comprise at least one humectant. Humectants useful herein include polyhydric alcohols such as glycerin, sorbitol, xylitol or low molecular weight PEGs, alkylene glycol such as polyethylene glycol or propylene glycol, or combinations thereof. In various embodiments, humectants are operable to prevent hardening of paste or gel compositions upon exposure to air. In various embodiments humectants also function as sweeteners.

In some embodiments, the humectant is present in the amount of about 1 to about 40% each by weight. In some embodiments, the humectant is sorbitol. In some embodiments, sorbitol is present at a concentration of from about 5% to about 25%, by weight. In some embodiments, sorbitol is present at a concentration of from about 5% to about 15%, by weight. In some embodiments, sorbitol is present at a concentration of about 10%, by weight. Reference to sorbitol herein refers to the material typically as available commercially in 70% aqueous solutions. In some embodiments, the total humectant concentration is from about 1 to about 60%, by weight. In some embodiments, the humectant is glycerin. In some embodiments, glycerin is present at a concentration of from about 5% to about 15%, by weight. In some embodiments, glycerin present is at a concentration of about 7.5%, by weight. In some embodiments, the humectant is propylene glycol. In some embodiments, propylene glycol is present at a concentration of about 5% to about 15%, by weight. In some embodiments, propylene glycol is present at a concentration of about 7%, by weight.

In some embodiments, the compositions comprise at least one cellulosic polymer such as hydroxyalkyl methyl celluloses (such as hydroxypropyl methyl cellulose, hydroxybutyl methyl cellulose, hydroxyethyl methyl cellulose, hydroxymethyl methyl cellulose and hydroxyethylpropyl methyl cellulose); carboxyalkyl methylcelluloses (such as carboxypropyl methyl cellulose, carboxybutyl methyl cellulose, carboxyethyl methyl cellulose, carboxymethyl methyl cellulose and carboxyethylpropyl methyl cellulose); hydroxyalkyl celluloses (such as hydroxypropyl cellulose, hydroxybutyl cellulose, hydroxyethyl cellulose, hydroxymethyl cellulose and hydroxyethylpropyl cellulose); alkyl celluloses (such as propyl cellulose, butyl cellulose, ethyl cellulose, methyl cellulose); carboxyalkyl celluloses (such as carboxypropyl cellulose, carboxybutyl cellulose, carboxyethyl cellulose, carboxymethyl cellulose and carboxyethylpropyl cellulose), and combinations thereof. In some embodiments, the cellulosic polymer comprises carboxymethyl cellulose.

In some embodiments, the compositions comprise at least one gum polymer, such as carrageenan gum, xanthan gum, and combinations thereof. In some embodiments, the gum polymer comprises xanthan gum.

Some embodiments comprise at least one polyacrylate polymer or co-polymer, such as a carbomer, and combinations thereof. In some embodiments, the polyacrylate polymer or co-polymer is selected from homo- and copolymers of acrylic acid crosslinked with a polyalkenyl polyether. Synthetic high molecular weight polymers of acrylic acid known as carbomer may be homopolymers of acrylic acid, crosslinked with an allyl ether pentaerythritol, allyl ether of sucrose or allyl ether of propylene, or combinations thereof. Carbomer has a USP classification of “carbomer homopolymer Type A”. Carbomers have the ability to adsorb, retain water and swell to many times their original volume. Carbomers codes (910, 934, 940, 941, 971, 974 and 934P) are an indication of molecular weight and the specific components of the polymer. Carbomers are commercially available, under the trade name Carbopol® from Lubrizol and other companies.

Some embodiments provide a composition obtained or obtainable by combining the ingredients as set forth in any of the embodiments described herein.

In some embodiments, the composition is in the form selected from a mouthwash, mouthrinse, mousse, foam, mouth spray, lozenge, tablet, dental implement, and a pet care product. In some embodiments, the composition is a mouthwash or mouthrinse.

Some embodiments of the present invention provide aqueous compositions comprising the following ingredients by weight:

| | Concentration |
|---|---|
| Ingredient | Range % wt/wt |
| Water | 50-90 |
| Humectants | 1-25 |
| Surfactant | 0.01-10 |
| Preservative | 0.01-1 |
| Flavor | 0.01-1 |
| Cellulosic polymer | 0.01-0.5 |
| Gum polymer | 0.01-0.5 |
| Polyacrylate polymer or co-polymer | 0.01-0.5 |
| Sodium fluoride | 0-0.05 |
| Ethyl alcohol | 0-8 |
| Sweetener | 0.01-0.5 |
| Cetylpyridinium chloride | 0.01-1 |
| Compound of Formula (II) | 0.01-1 |

Some embodiments provide a method of treating halitosis comprising administering any embodiment of the present invention to the oral cavity of a subject in need thereof.

Some embodiments comprise at least one colorant. Colorants such as dyes may be food color additives presently certified under the Food Drug & Cosmetic Act for use in food and ingested drugs, including dyes such as FD&C Red No. 3 (sodium salt of tetraiodofluorescein), Food Red 17, disodium salt of 6-hydroxy-5-{(2-methoxy-5-methyl-4-sulphophenyl)azo}-2-n-aphthalenesulfonic acid, Food Yellow 13, sodium salt of a mixture of the mono and disulphonic acids of quinophtalone or 2-(2-quinolyl)indanedione, FD&C Yellow No. 5 (sodium salt of 4-p-sulfophenylazo-1-p-sul-fophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-δ-3,5-cyclohexadienimine], FD&C Blue No. 1 (disodium salt of dibenzyldiethyldiamino-triphenylcarbinol trisulfonic acid anhydrite), FD&C Blue No. 2 (sodium salt of disulfonic acid of indigotin) and mixtures thereof in various proportions. Typically, colorants if included are present in very small quantities.

At least one flavor agents may also be included in some embodiments of the present invention. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavors include mints such as peppermint, artificial vanilla, cinnamon derivatives, and various fruit flavors, whether employed individually or in admixture. Generally, any flavoring or food additive, such as those described in Chemicals Used in Food Processing, publication 1274 by the National Academy of Sciences, pages 63-258, may be used. Typically, flavorants if included are present at about 0.01-1%, by weight. In some embodiments, flavoring may be present in about 0.2%, by weight.

Some embodiments include at least one sweetener, including both natural and artificial sweeteners. Suitable sweetener include water soluble sweetening agents such as monosaccharides, disaccharides and poysaccharides such as xylose, ribose, glucose (dextrose), mannose, galactose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalaine methyl ester (aspartame), or combinations thereof. In general, the effective amount of sweetener is utilized to provide the level of sweetness desired for a particular composition, will vary with the sweetener selected. This amount will normally be about 0.001% to about 5% by weight of the composition. In some embodiments, the sweetener is sodium saccharin and present at about 0.01% by weight of the composition.

At least one optional breath freshening agent may be provided. Any orally acceptable breath freshening agent can be used, including without limitation zinc salts such as zinc gluconate, zinc citrate and zinc chlorite, alpha-ionone and mixtures thereof. One or more breath freshening agents are optionally present in a breath freshening effective total amount.

Optionally, the composition may include at least one tartar control (anticalculus) agent. Tartar control agents among those useful herein include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts, or combinations thereof. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J.

In some embodiments, tartar control agent is present at a concentration of from about 0.01% to 10%, by weight. In some embodiments, the tartar control agent is present at a concentration of about 1%, by weight. In some embodiments, the tartar control agent also acts as a buffer. For example, in a phosphate buffer system, sodium phosphate monobasic is present at a concentration of from about 0.01% to about 5%, by weight, and disodium phosphate is present at a concentration of from about 0.01% to about 5%, by weight, the precise ratio depending upon the other excipients in the formulation and the desired pH.

In some embodiments, at least one antioxidant is optionally added. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Also optional, saliva stimulating agent, useful for example in amelioration of dry mouth may be included. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective total amount.

In some embodiments, at least one antiplaque (e.g., plaque disrupting) agent may be included. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and mixtures thereof.

In some embodiments, at least one optional desensitizing agent, including potassium citrate, potassium chloride, potassium tartrate, potassium bicarbonate, potassium oxalate, potassium nitrate, strontium salts, and mixtures thereof, are included.

In some embodiments, the pH of the composition is between about pH 1 and about pH 9. In some embodiments the pH of the composition is between about pH 2 and about pH 8. In some embodiments, the pH of the composition is between about pH 2 and about pH 7. In preferred embodiments, the pH of the composition is between about pH 2 and about pH 6. In more preferred embodiments, the pH of the composition is between about pH 2 and about pH 5.

In some embodiments, the methods comprise the step of rinsing the oral cavity with a composition as described herein. In some embodiments, 5 ml or more of the composition is gargled. In some embodiments, 10 ml or more is used. In some embodiments, 10-50 ml is used. In some embodiments, 15-25 ml or more is used. In some embodiments, 15 ml or more is used. In some embodiments, the individual gargles with the composition multiple times per day. In some embodiments, the individual gargles with the composition on multiple days. In some embodiments, the individual gargles with the composition every 4 to 6 hours up to 6 times per day.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### EXAMPLES

### Example 1

Table 1 (below) describes the formulation for two exemplary compositions of the present invention (Composition I and Composition II).

TABLE 1

| | |
|---|---|
| Composition I | Composition II |

| | |
|---|---|
| Ingredient | Wt. % |

| | | |
|---|---|---|
| Sucralose | 0.02 | 0.02 |
| Sodium Fluoride | 0.05 | 0.05 |
| Sodium Benzoate | 0.11 | 0.11 |
| Glycerin | 7.5 | 7.5 |
| Sorbitol | 5.5 | 5.5 |
| Propylene Glycol | 5 | 5 |
| Pluronic F127 | 0.15 | 0.15 |
| Ethyl Alcohol | — | 6 |
| Water | 81.57 | 75.57 |
| Cetylpyridinium chloride | 0.05 | 0.05 |
| Compound of Formula (II) | 0.05 | 0.05 |
| Total | 100 | 100 |

### Example 2

### Exemplary Method of Preparation

Some embodiments of the present invention can be prepared according to the following procedure. A pre-mix is prepared by adding propylene glycol to a container and adding menthol thereto. The combination is mixed until the menthol is dispersed. Flavor is added and mixed for about 3 minutes. Water is then added to the main mixer and the mixer is turned on. Pluronic is then added until it is sufficiently dispersed. Saccharin, potassium sorbate and a compound of Formula (II) are then added to the main mixer and mixed for about 3 minutes. Citric acid is added to the main mixer and mixed for about 5 minutes. Glycerin is added to the main mixer. Sorbitol is added to the main mixer and the mixed for about 5 minutes. The pre-mix is then added to the main mixer and mixed for about 15 minutes.

### Example 3

To evaluate minimum inhibitory concentrations (MICs), the concentration of the twenty four hour culture in log phase is adjusted by diluting in tryptic soy broth (TSB) so that an optical density of 0.2 at 610 nm is obtained. The bacterial culture is then ready to be used for testing.

Three solutions are prepared: (1) 1% CPC solution in ethanol; (2) 1% solution of the Compound of Formula (II) in water; and (3) 0.5% of a Compound of Formula (II) +0.5% CPC. The solutions are diluted 1:9 in TSB. They are added to a 96-well plate and a serial dilution (2-fold) is made across the plate. The bacterial inoculum at 0.2 OD, 100 μL is added to every well. The plate is incubated overnight and read on a plate reader the following day.

TABLE 2

| Active | MIC (ppm) |
|---|---|
| Negative Control | >250 |
| Compound of Formula (II) | 0.49 |
| CPC | 0.98 |
| Compound of Formula (II) + CPC | <0.12 |

0.5% CSA-13+0.5% CPC is <0.4=synergistic Example 4

Water is used as a negative control. 0.04 mL of each sample is added into a GC headspace vial with each sample being tested in duplicate. A saliva inoculum is prepared using 65% whole saliva collected after lunch, 30% deionized water, and 5% of FTG media. Three milliliters of the saliva mixture is added to the vials containing the samples. The vials containing the saliva mixture and samples are capped.

The capped vials are incubated overnight at 37° C. in a water bath with shaking. Gas chromatography (GC) is utilized to determine the amount of reduction of volatile sulfur compounds (VSC) as compared with the negative control.

The data described in Table 3 (below) demonstrates that compositions of the present invention are effective in reducing volatile sulfur compounds, and would therefore likely be effective in treating oral malodor.

TABLE 3

| Formulation | Reduction in VSC |
|---|---|
| Negative Control | 13.1 |
| 0.05% Compound of Formula (II) | 97 |

### Example 4

In this assay, two fluorescent dyes are used to give a rapid measure of bacteria viability.

A sample from a mixed species bacterial chemostat culture, OD˜0.6 is transferred to sterile 1.5 mL microcentrifuge tubes and centrifuged for 10 min at 12,000×g to pellet the bacteria. Bacteria are then resuspended in 100 μL of sterile phosphate buffered saline (PBS). Samples are treated with 100 μL (high dose) or 20 μL (low dose) of mouthwash or control solution. Killing is stopped after 30 seconds, as indicated by the addition of 1.35 mL of D/E Neutralization Buffer (Invitrogen). Samples are centrifuged for 10 min at 12,000×g to pellet bacteria and pellets are resuspended in 500 μL of sterile PBS to wash, then centrifuged again. Finally samples are suspended in 150 μL of sterile PBS and 50 μL aliquots are transferred to each of three wells of sterile 96-well plates, which are subjected to bacteria staining using Invitrogen BacLight Live/Dead bacterial viability kit. 50 μL of 2× solution containing two dyes (SYTO9 dye [green] and propidium iodide [red]) are added to samples in the 96-well plates. Plates are incubated for 15 min at room temperature, protected from light and subjected to fluorescence reading at excitation wavelength 485 nm and emission wavelength 535 and 635 nm. Results are presented as a percentage of cells that are viable relative to a control sample treated with PBS.

Table 4 (below) describes data demonstrating that the compositions of the present invention provide a synergistic antimicrobial effect after 30 seconds of use.

TABLE 4

| | Log Reduction at |
|---|---|
| Formulation | 30 seconds |
| 0.05% CPC | 3.93 |
| 0.05% Compound of Formula (II) | 0.38 |
| 0.05% Compound of Formula (II) + 0.05% CPC | 7.52 |

### Example 5

The anti-inflammatory activity of a compound of Formula (II) is studied against six human inflammatory cytokines, PGE₂, IL-1β, IL-6, IL-8, TNF-α, and GM-CSF in separate sets of experiments; first against PGE2, then against the five other cytokines. Human monocyte U937 cells are differentiated to macrophages and then generation of cytokines is induced by stimulating the cells using heat killed P. gingivalis (HKPG, 1×10⁸ cells). Different types of inflammatory cytokines are generated by U973 cells and released into the supernatant.

A compound of Formula (II) displays higher anti-inflammatory efficacy against three cytokines, IL-1β, IL-6, and TNF-α. Specifically, at a concentration of 0.1 ppm, a compound of Formula (II) reduces 21% of IL-1β, 60% of IL-6 and 80% of TNF-α.

### Example 6

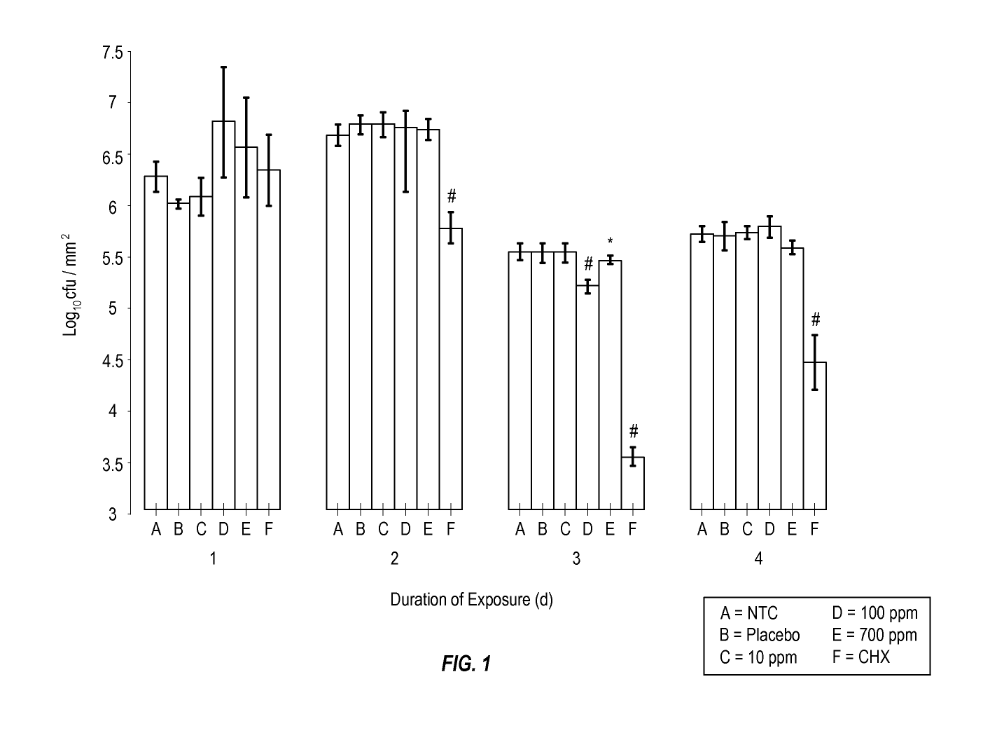

The efficacy of CSAs was also examined relative to a commercially available chlorhexidine rinse. Plaques were treated (and sampled) daily with mouthwash solutions containing 0, 10, 100, and 700 ppm CSA-13 (structure shown below). A formulation containing chlorhexidine (CHX) was used as the positive control. Treated disks were sampled for viable anaerobic organisms (in triplicate); viable organisms enumerated after a six day incubation. Significant differences compared to placebo-treated-plaques are indicated in the chart depicted in FIG. 1 (*, p<0.05; #, p<0.001, n=9). NTC is the untreated control; placebo is the mouthrinse formulation without CSA.

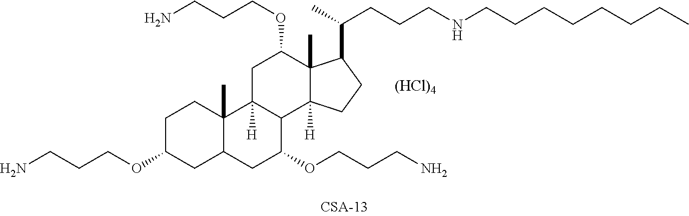

### Example 7

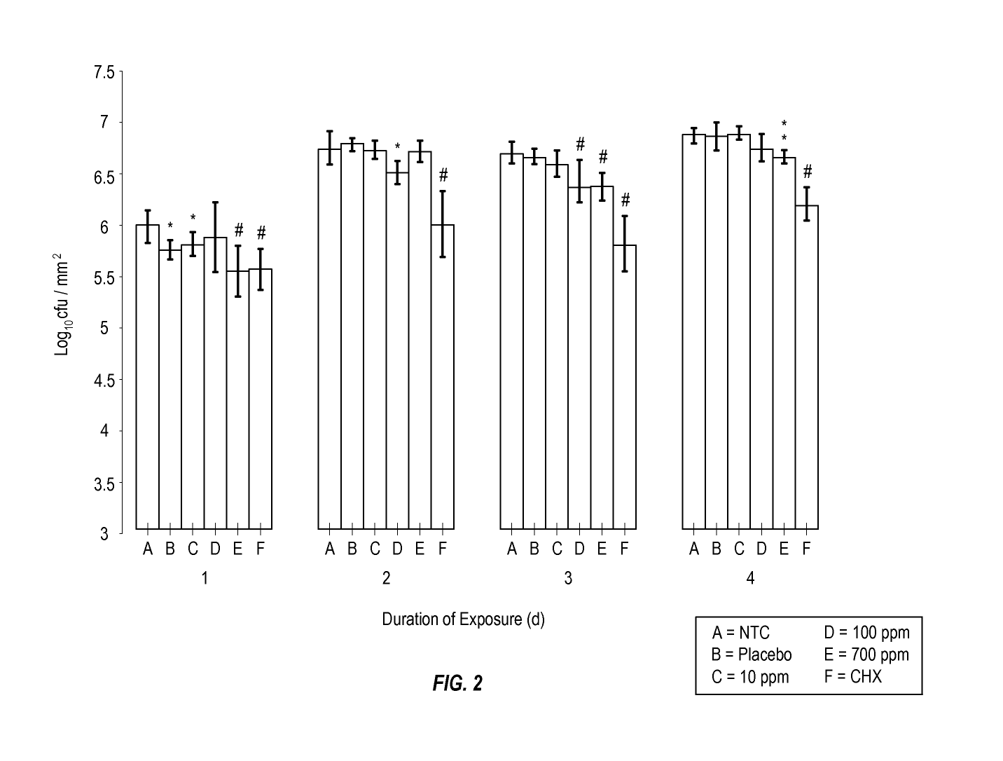

The efficacy of CSAs was further examined relative to commercially available mouth rinses with chlorhexidine (CHX) and cetylpyridinium chloride (CPC). Plaques were treated (and sampled) daily with mouthwash solutions containing 0, 100, and 700 ppm CSA-13 (structure shown above). Formulation containing CHX and CPC were used as positive controls. Treated disks were sampled for viable anaerobic organisms (in triplicate); viable organisms enumerated after a six day incubation. Significant differences compared to placebo-treated-plaques are indicated in the chart depicted in FIG. 2 (*, p<0.05; #, p<0.001, n=9). NTC is the untreated control; placebo is the mouthrinse formulation without CSA.

### Example 8

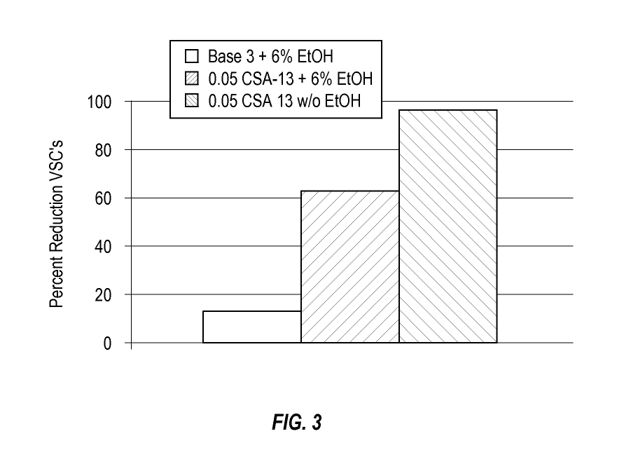

CSA-13 is also effective at reducing the levels of volatile sulfur compounds (VSCs) generated from odor-producing salivary bacteria in an in vitro mouth odor VSC assay. CSA-13 was incubated overnight with odor-producing salivary bacteria and the VSCs were measured. The results are shown in the chart depicted in FIG. 3, from left to right.

What is claimed is:

1. A composition, comprising
an aqueous oral care formulation, the oral care formulation further comprising at least one cationic steroidal compound.

2. The composition of claim 1, wherein the oral care formulation is selected from the group consisting of mouthwash, mouthrinse, toothpaste, mousse, foam, and mouth spray.

3. The composition of claim 2, wherein the composition is a mouthwash.

4. The composition of claim 3, wherein the mouthwash is substantially free of ethanol.

5. The composition of claim 1, wherein the at least one cationic steroidal compound is a compound of Formula (I): 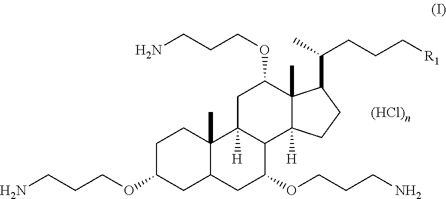
wherein R₁ is selected from —OH and NH—R₂, R₂ is C₂-C₁₄ alkyl, C₂-C₁₄ alkenyl, or C₂-C₁₄ akynyl, and n is 3 or 4.

6. The composition of claim 5, wherein the compound of Formula (I), is selected from the group consisting of

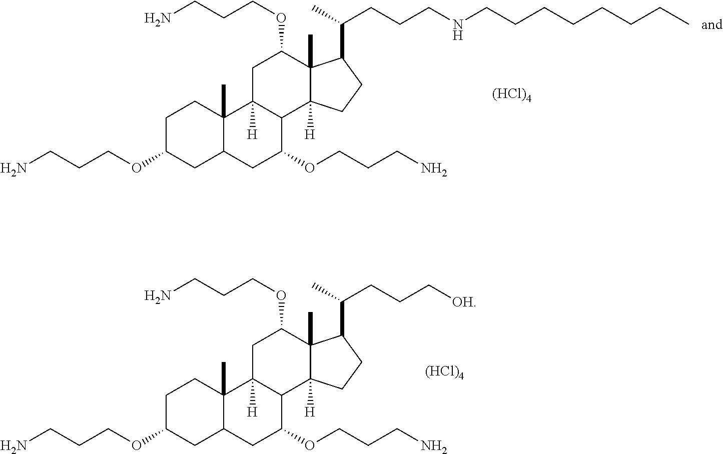

7. The composition of claim 6, wherein cationic steroidal compound is

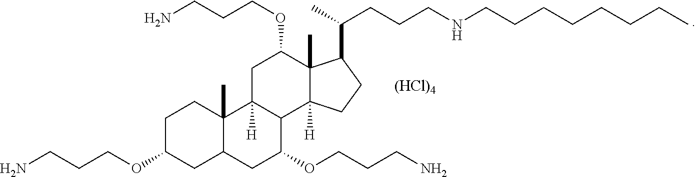

8. The composition of claim 1, wherein the cationic steroidal compound is present at a concentration of from about 0.01% to about 0.1%, by weight, of the composition.

9. The composition of claim 8, wherein the cationic steroidal compound is present at a concentration of about 0.05%, by weight, of the composition.

10. The composition of claim 1, further comprising a fluoride ion source; wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

11. The composition of claim 10, wherein the fluoride ion source comprises sodium fluoride.

12. The composition of claim 1, further comprising at least one humectant.

13. The composition of claim 12, wherein the at least one humectant is selected from the group consisting of glycerin, sorbitol, xylitol, polyethylene glycol, propylene glycol, and combinations thereof.

14. The composition of claim 1, further comprising at least one cellulosic polymer.

15. The composition of claim 14, wherein the at least one cellulosic polymer is selected from the group consisting of hydroxypropyl methyl cellulose, hydroxybutyl methyl cellulose, hydroxyethyl methyl cellulose, hydroxymethyl methyl cellulose, hydroxyethylpropyl methyl cellulose, carboxypropyl methyl cellulose, carboxybutyl methyl cellulose, carboxyethyl methyl cellulose, carboxymethyl methyl cellulose, carboxyethylpropyl methyl cellulose, hydroxypropyl cellulose, hydroxybutyl cellulose, hydroxyethyl cellulose, hydroxymethyl cellulose and hydroxyethylpropyl cellulose, propyl cellulose, butyl cellulose, ethyl cellulose, methyl cellulose, carboxypropyl cellulose, carboxybutyl cellulose, carboxyethyl cellulose, carboxymethyl cellulose, carboxyethylpropyl cellulose, and combinations thereof.

16. The composition of claim 1, further comprising at least one gum polymer.

17. The composition of claim 16, wherein the at least one gum polymer is selected from the group consisting of carrageenan gum, xanthan gum, and combinations thereof.

18. The composition of claim 1, further comprising a polyacrylate polymer or polyacrylate co-polymer.

19. The composition of claim 18, wherein the polyacrylate polymer or polyacrylate co-polymer formed from monomers selected from the group consisting of acrylic acid, polyalkenyl polyether, allyl ether pentaerythritol, sucrose allyl ether, and propylene allyl ether.

20. The composition of claim 1, further comprising at least one agent selected from the group consisting of flavoring agents, sweeteners, colorants, tartar control agents, breath freshening agents, saliva stimulating agents, antioxidants, antiplaque agents, desensitizing agents, and combinations thereof.

21. A composition, comprising a substantially non-aqueous oral care formulation, the oral care formulation further comprising at least one cationic steroidal compound.

22. The composition of claim 21, wherein the oral care formulation is selected from the group consisting of lozenge, tablet, dental implement, orthodontic device, flexible strip, and chewing gum.

23. The composition of claim 21, wherein the cationic steroidal compound is carried by a flexible strip, the strip further comprising a first side and a second side, and wherein at least side comprises an adhesive.

24. The flexible strip of claim 23, wherein the strip comprises a matrix comprising a fast-dissolving material.

25. The flexible strip of claim 23, wherein the strip comprises a slow-release mucoadhesive device.

26. The composition of claim 22, wherein the oral care formulation is in an orthodontic device.

27. The composition of claim 26, wherein the orthodontic device is selected from the group consisting of a removable retainer, a temporary retainer, a permanent retainer, braces brackets, braces wires, braces fasteners, a bridge, and tooth spacers.

28. The composition of claim 21, wherein the at least one cationic steroidal compound is a compound of Formula (I): 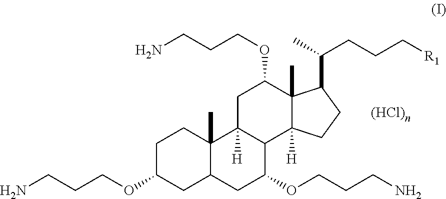
wherein R₁ is selected from —OH and NH—R₂, R₂ is C₂-C₁₄ alkyl, C₂-C₁₄ alkenyl, or C₂-C₁₄ akynyl, and n is 3 or 4.

29. The composition of claim 28, wherein the compound of Formula (I), is selected from the group consisting of

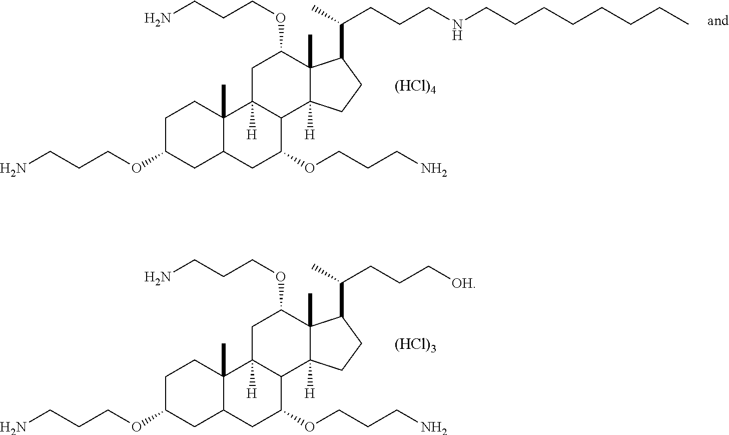

30. The composition of claim 29, wherein cationic steroidal compound is

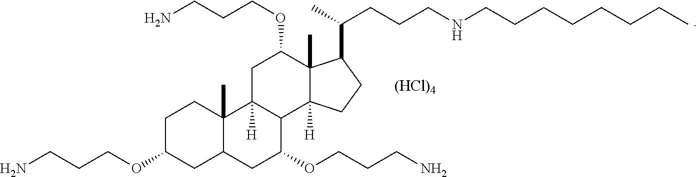

31. The composition of claim 21, wherein the cationic steroidal compound is present at a concentration of from about 0.01% to about 0.1%, by weight, of the composition.

32. The composition of claim 31, wherein the cationic steroidal compound is present at a concentration of about 0.05%, by weight, of the composition.

33. The composition of claim 21, further comprising a fluoride ion source; wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

34. The composition of claim 33, wherein the fluoride ion source comprises sodium fluoride.

35. A method of treating a disease or condition of the oral cavity comprising
identifying a subject with a disease or condition of the oral cavity; and
administering a composition comprising a cationic steroidal compound to the oral cavity of the subject in need thereof.

36. The method of claim 35, wherein the disease or condition of the oral cavity is selected from the group consisting of gingivitis, periodontitis, and caries.

37. A method of treating an inflammatory condition of the oral cavity comprising
identifying a subject with an inflammatory condition of the oral cavity; and
administering a composition comprising a cationic steroidal compound to the oral cavity of a subject in need thereof.

38. A method of treating halitosis comprising
identifying a subject with halitosis; and
administering a composition comprising a cationic steroidal compound to the oral cavity of a subject in need thereof.

39. The method of claim 38, further comprising reducing volatile sulfur compounds in oral cavity of a subject in need thereof.

40. The method of claim 39, wherein the level of at least one of hydrogen sulfide, methyl mercaptan, or dimethyl sulfide in the oral cavity is reduced by at least 25%.

41. The method of claim 39, wherein the reduction in volatile sulfur compounds in the oral cavity of a subject in need thereof is not accomplished by oxidation.

42. A method of preventing halitosis comprising
identifying a subject at risk for developing halitosis; and
administering a composition comprising a cationic steroidal compound to the oral cavity of a subject in need thereof.

43. The method of claim 42, further comprising reducing volatile sulfur compounds in oral cavity of a subject in need thereof.

44. The method of claim 43, wherein the level of at least one of hydrogen sulfide, methyl mercaptan, or dimethyl sulfide in the oral cavity is reduced by at least 25%.

45. The method of claim 43, wherein the reduction in volatile sulfur compounds in the oral cavity of a subject in need thereof is not accomplished by oxidation.

46. The composition comprising a compound of Formula (I): 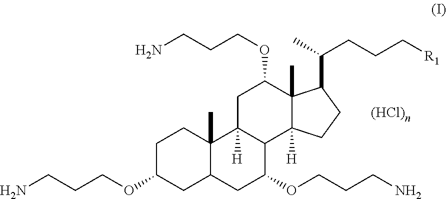
wherein R₁ is selected from —OH and NH—R₂, wherein R₂ is C₂-C₁₄ alkyl, C₂-C₁₄ alkenyl, or C₂-C₁₄ akynyl, and n is 3 or 4.
